(19) United States
(12) Patent Application Publication
(10) Pub. No.: US 2016/0374863 A1
(43) Pub. Date: Dec. 29, 2016

(54) See-Through Wound or Skin Condition Dressing and Treatment System

(71) Applicant: Alfred J. LaGreca, Hingham, MA (US)

(72) Inventor: Alfred J. LaGreca, Hingham, MA (US)

(21) Appl. No.: 14/820,724

(22) Filed: Aug. 7, 2015

Related U.S. Application Data
(63) Continuation of application No. PCT/US2014/015282, filed on Feb. 7, 2014.
(60) Provisional application No. 61/761,992, filed on Feb. 7, 2013.

(51) Int. Cl. A61F 13/02

(52) U.S. Cl. CPC A61F 13/025; A61F 13/0233; A61F 13/0253; A61F 13/0266; A61F 2013/0057

(57) ABSTRACT

A reclosable wound or skin condition dressing and treatment system (10). The system (10) has a base portion (20) that has a circumference and lower (22) and upper (24) sides, the lower side (22) carrying a first adhesive that is constructed and arranged to adhere to the skin (S), and the upper side (24) defining an adhesive release surface. There is a covering portion (30) that has a second adhesive on its lower side (32) that releasably engages with the release surface (24) of the base portion (20), and an upper side (34) that is a semi-permeable layer. The covering portion (30) overlies at least part of and engages with the upper side (24) of the base portion (20). The semi-permeable layer (34) of the covering portion (30) fully covers the open area (V) of the base portion (20), so as to cover a wound or skin condition (W) located in the open area (V). The semi-permeable layer (34) of the covering portion (30) is clear or translucent so that a caregiver can see through it to observe the skin, wound, skin condition (W) or dressing below.

### CROSS-REFERENCE TO RELATED APPLICATIONS

This application is a continuation of International Application Number PCT/US2014/015282 filed on Feb. 7, 2014, which itself claims priority of U.S. Provisional Application 61/761,992, filed on Feb. 7, 2013. Priority of both of these applications is claimed herein.

### FIELD

This disclosure relates to a dressing for wounds or skin conditions.

### BACKGROUND

Wound dressings can need to be removed so that the caregiver can check the condition of the wound. The dressing then needs to be replaced. Since the dressings are adhered to the skin, such repeated removal of dressings can damage the skin, cause pain, and lead to infections and other side effects.

### SUMMARY

A purpose of the disclosure is to reduce the number of adverse events caused by the time required for nurses, doctors and patients to remove taped dressings from wounds for evaluation or for changing, as well as to reduce the negative factors related to the removing of the tape. The removal of tape, over the time required for wounds to heal, can cause scaring, infections and dermatitis. Further, the necessary repeated removal is very painful. The 24/7 observation of incisions or wounds without exudates allowed by the transparent cover member can significantly reduce the severity of infections. Also, this wound observation takes place while the wound remains sterile under the transparent cover member. There is thus no additional risk of acquiring a hospital acquired infection (HAI) as can happen when a dressing is lifted or removed so that the wound can be inspected. The see-through nature of the present dressing can lead to a significant reduction in HAIs, which currently cost billions of dollars per year.

A significant cause of HAIs is air-borne pathogens that infect a wound during a dressing change. The present see-through dressing system helps to protect the patient from HAIs, as in many cases because the wound can be inspected under aseptic conditions, the dressing can remain on during the entire hospital stay. Since there are fewer deleterious pathogens in the home environment, changing the dressing at home may reduce the risk of infections.

The disclosure has at least the following benefits:
Reduces hospital costs by reducing the time it takes a nurse or doctor to change a bandage. This reduces the effectiveness of the medical staff which in turn leads to adverse events such as infections, wrong medications etc. Thousands of patients die each year due to adverse events, most caused by the shortage of medical personnel.
Reduces disposal of medical waste.
Increases home care patient independence.
Reduces unnecessary bandage changes as wound and dressing can be evaluated without removal of bandage.
May reduce infection from bacteria.
Reduces pain, scarring and dermatitis caused by the daily changing of a bandage held to the skin with tape.
Allows bandage to be changed and wound to be evaluated 24/7 or dressed as frequently as desired without irritation that is caused by tape removed from the skin. This allows hospital acquired infections to be identified and treated earlier, which can reduce the seriousness and associated costs.
Improves the quality of wound management as the type of dressing/medication may be changed without having to remove the base of the bandage.
Decreases risk of skin conditions such as skin irritations, an example being bed sores, from becoming ulcers. This is because the skin condition may be treated prior to an ulcer or wound forming.
Allows skin tears (which are prevalent in the elderly) to be managed in a sterile, observable environment.

This disclosure features in one example a reclosable wound or skin condition dressing and treatment system comprising a base portion having a circumference and lower and upper sides, the lower side carrying a first adhesive that is constructed and arranged to adhere to the skin, the upper side defining an adhesive release surface, and a covering portion having a second adhesive on its lower side that releasably engages with the release surface of the base portion, and an upper side comprising a semi-permeable layer, the covering portion overlying at least part of and engaging with the upper side of the base portion, wherein the semi-permeable layer of the covering portion fully covers the open area of the base portion, so as to cover a wound or skin condition located in the open area. The semi-permeable layer of the covering portion is clear or translucent so that a caregiver can see through it to observe the skin, wound, skin condition or dressing below.

The first adhesive may comprise a hydrocolloid adhesive. The second adhesive may comprise a pressure-sensitive tape. The base portion may circumscribe a wound or skin condition. The covering portion may overly essentially all of the base portion. The covering portion may define a pull tab adapted to be gripped by a user to assist in separating the covering portion from the base portion. The system may further comprise a removable protective cover over the first adhesive on the lower side of the base portion.

Also featured is a reclosable wound or skin condition dressing and treatment system comprising a covering portion having a lower side that defines an annular adhesive and an upper side comprising a clear or translucent semi-permeable layer, the covering portion spanning the opening of the annulus such that it fully covers the open area of the annulus, to cover a wound or skin condition located in the open area while keeping the wound or skin condition visible through the covering portion.

The adhesive may comprise a hydrocolloid adhesive. The covering portion may circumscribe a wound or skin condition. The covering portion may define a pull tab adapted to be gripped by a user to assist in separating the covering portion from the skin. The system may further comprise a removable protective cover over the adhesive.

### BRIEF DESCRIPTION OF THE DRAWINGS

Other objects, features and advantages will occur to those skilled in the art from the following description of the preferred embodiments, and the accompanying drawings, in which:

FIG. 1 is a cross-sectional view of a two-piece wound dressing system in place surrounding a wound or skin condition.

FIG. 2 is a cross-sectional view of a one-piece wound dressing system in place surrounding a wound or skin condition.

FIG. 3 is a top view of the one-piece wound dressing system in place surrounding a wound or skin condition, shown in FIG. 2.

### DETAILED DESCRIPTION OF EXAMPLES

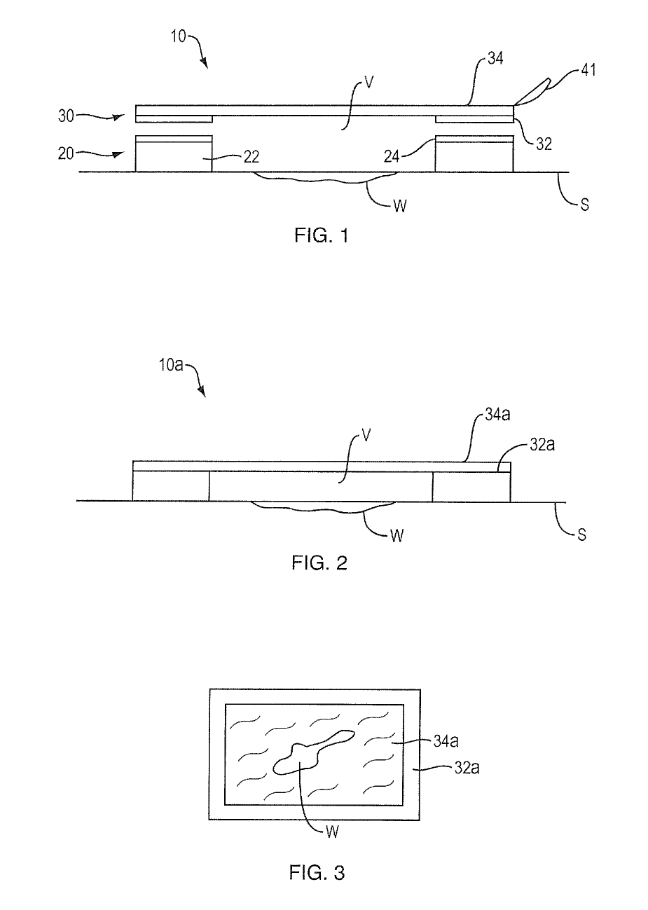

This invention may be accomplished in a disposable or reclosable wound or skin condition dressing and treatment system. One non-limiting example of the inventive wound or skin condition dressing and treatment system 10, FIG. 1, has two separable components: a base member 20 that is adhered to skin surface S, and a cover or top member 30 that is removably adhered to the top of base member 20. A bandage, dressing pad or treatment pad itself can be but does not need to be held in place against the skin.

The base member 20 is an annulus or other shape that defines or encompasses an open area. The base member is semi-permanently adhered to the skin, with the wound or skin condition “W” being treated located within the open area such that the base surrounds the wound or skin condition, preferably without touching it. Member 20 can comprise an annulus 22 of a hydrocolloid with an adhesive on the lower side that is covered with a removable protective covering (not shown) that is removed just before use. Top layer 24 of base member 20 can be a polyethylene film (or another plastic from which an adhesive can be released) that is the same size and shape as annulus 22 and is adhered to the top of annulus 22. The base member provides the function of a means to removably couple the top member to the skin, while the top member may be spaced from the skin so as not to disturb the wound.

The adhesion of the base member to the skin may be accomplished with any known adhesive that is compatible with skin. The adhesive may be that used on NEXCARE absolute waterproof first aid tape by the 3M Company, or a hydrocolloid adhesive such as that disclosed in U.S. Pat. No. 4,952,618, for example, or foam tape from 3M. These adhesives may remain in contact with the skin for five to ten days under normal use, including movement and exposure to water, without loosening and without substantial negative impact to the skin. In an embodiment, tape is not used and the base member remains on the skin for multiple days. This obviates the need for frequent removal of tape from the skin that leads to so many problems in the current wound-care regimen.

The base can have a desired, preferably annular shape, such as a circular, oval or rectangular annulus, for example. The adhesive on the lower side of the base is used to secure the base to the person's skin, surrounding the wound or area of the skin requiring covering or treatment. Adhesives that can be used to couple the base to the skin can also include hydrocolloid material such as 3M #9943 or #9944, or Duoderm by Convatec of Skellman, N.J., or an adhesive hydrogel such as available from Scapa North America in Windsor, Conn. The adhesive would normally be covered with a removable protective layer as is commonly used to protective the sticky side of an adhesive tape, label or sticker. The base is preferably sterile, and can be made with anti-bacterial material.

Top member 30 can be of the same size and perimeter shape as the base member, except that it is not annular. Accordingly, member 30 covers base member 20, and also spans the opening defined by the base member, to enclose the volume “V” in which wound or skin condition W is located, and also enclose a dressing or treatment pad should such be used. Top member 30 is preferably constructed with an annulus 32 of polyurethane medical grade adhesive tape that overlies some or all of base annulus 22. Top member 30 also includes a semi-permeable sheet or membrane 34. Sheet 34 may be but need not be a polyurethane membrane, one example of which is 3M #9833. Sheet 34 may be secured to annulus 32. Sheet 34 spans the annular opening of base 20. Sheet 34 is preferably adhesive-free so that it does not adhere to the wound or any dressing that touches sheet 34. Top member 30 is sterile and can be made of anti-bacterial material.

Sheet 34 is preferably translucent or clear. Sheet 34 may also be hydrophobic. If sheet 34 is clear or translucent, the pad or the underlying skin region can be inspected without separating the top member from the bottom member.

The subject system is a very low-profile wound covering that can stay in place for many days, thus decreasing the need to pull tape off of the skin in order to view a wound, or change a bandage or treatment pad. The result is less patient pain and suffering, and decreased costs associated with treating infections caused by repeated tape removal, which is prevalent in chronic-care situations in which a dressing may need to be changed multiple times per day, for a week or more. This also decreases medical costs by changing dressings only when necessary, decreased waste and decreased labor costs associated with removing tapes and their residue. Increasing nursing and medical availability caused by this invention will decrease adverse events causing complications and sometimes death. Also, the invention allows for the continuous observation of an incision or wound without exudates. This allows infections, swelling, redness or bursting and other disorders to be identified immediately, which can lead to a significant reduction of the severity of infections and the associated costs.

### Alternative Materials

The subject system contemplates a variety of means for adhesively coupling the base member to the skin of a person or animal, a variety of means for removably coupling the top member to the base member, and a variety of wound and skin condition treatment modalities held in place by the base and top members.

For example, the base can be coupled to the skin with any adhesive that accomplishes sufficient adhesion along with the benefits of less skin irritation or damage. An additional example is adhesive silicone gels such as those manufactured and used by Scapa, Windsor, Conn. Also, the inventive dressing can be removably coupled to the skin by an elastic compression wrap bandage overlaying the inventive dressing (such as an Ace bandage from BD, Franklin Lakes, N.J.) and wrapped around a portion of the body such as a limb or the torso.

As another example, the top can be coupled to the base by an adhesive on the bottom side of the top member that is sufficient to hold the top on the base but allows the top to be released from the base and removed. The top can then be placed back on the base at least once if desired, and preferably multiple times, over the up to 5-7 day typical useful life of the inventive device. One example of an adhesive that may be useful for this purpose is a medical grade pressure sensitive tape adhesive such as 3M #1510 on one part (e.g., on the bottom surface of the top member) and an adhesive-releasing plastic surface (e.g., a polyethylene film) on the other part (e.g., making up the top surface of the lower annulus).

There are many examples of substances that can make up or be carried by a pad, gauze or the like that can be held in place by the top member, and lie against the skin, and/or protrude into a wound in the skin. As the inventive system can be opened and reclosed, and allows the top to be replaced, the invention can be use to accomplish a desired treatment regimen for a wound, skin condition, or the like. As one example, the pad can carry or comprise or be entirely made from certain known materials that have known medical effects. Among these are:
Foam composites such as Mepilex Soft Silicon by MoLnlycke of Denmark. These products typically serve more than purpose, for example a combination which maintains moisture in the wound site, is a barrier to bacteria, and absorbs excess exudates.
Alginates such as Algicell Calcium Alginate by Derma Sciences of Princeton, N.J. These absorb excess exudates, maintain a moist healing environment, and facilitate autolytic debridement.
Silver alginate such as Maxsorb Extra by Medline Industries of Mundelsein, Ill. In addition to the benefits of the alginate, the silver reduces infections from a wide range of microorganisms.
Antimicrobial such as Anticoat 7 by Smith and Nephew of London, England. This can be an effective barrier to bacterial penetration that may reduce infections in partial or full thickness wounds.
Composites such as ThinSite Hydrogel by Swiss America of Waldenburg, Switzerland. These are made of a combination of wound dressings which improve healing, reduce infections and absorb exudates.
Transparent dressings such as Tegaderm #1628 by 3M allow the wound to be viewed at all times but have the disadvantage of difficulty in removal from the skin and wound. The invention significantly reduces the damage of removal but allows for continued viewing of the wound or skin condition or the like.
Hydrocolloids such as Dermatell by Gentell, Inc of Skellman, N.J. These support a moist healing environment and autolytic debridement and non traumatic dressing removal.
Hydrogels such as Ameriderm wound gel dressing by Ameriderm Labs Ltd. of Patterson, N.J. The gel's high glycerin content facilitates the natural wound healing process.
Silver such as Silverlon by Argentum Medical LLC of Willowbrook, Ill. reduces infections by resisting and eliminating most microorganisms.

Of course, the invention contemplates other materials, medications and the like, to accomplish any desired or custom treatment of a condition such as skin irritation caused by pressure prior to the wound developing, or a surface wound. The invention speeds and eases wound and skin care and treatment in situations in which the skin needs to be covered. The invention also decreases incidence of tape removal-related infections. The invention thus can reduce the costs associated with wound and skin care.

The disclosure also contemplates a single layer dressing 10a, FIGS. 2 and 3, which is essentially top member 30 from FIG. 1. Adhesive annulus 32a carries transparent sheet 34a, which spans the open area of the annulus. Annulus 32a can be placed directly on the skin. The result is a clear window through which the wound, or any dressing or the like held in place over the wound, can be continuously viewed.

Although specific features of the invention are shown in some figures and not others, this is for convenience only, as some features may be combined with any or all of the other features in accordance with the invention.

The use of any and all examples, or exemplary language (e.g., “such as”) provided herein, is intended merely to better illustrate the invention and does not pose a limitation on the scope of the invention.

A variety of modifications to the embodiments described herein will be apparent to those skilled in the art from the disclosure provided herein. Thus, the invention may be embodied in other specific forms without departing from the spirit or essential attributes thereof.

What is claimed is:

1. A reclosable wound or skin condition dressing and treatment system, comprising:
a base portion having a circumference and lower and upper sides, the lower side carrying a first adhesive that is constructed and arranged to adhere to the skin, the upper side defining an adhesive release surface; and
a covering portion having a second adhesive on its lower side that releasably engages with the release surface of the base portion, and an upper side comprising a semi-permeable layer, the covering portion overlying at least part of and engaging with the upper side of the base portion, wherein the semi-permeable layer of the covering portion fully covers the open area of the base portion, so as to cover a wound or skin condition located in the open area;
wherein the semi-permeable layer of the covering portion is clear or translucent so that a caregiver can see through it to observe the skin, wound, skin condition or dressing below.

2. The system of claim 1 wherein the first adhesive comprises a hydrocolloid adhesive.

3. The system of claim 1 wherein the second adhesive comprises a pressure-sensitive tape.

4. The system of claim 1 wherein the base portion circumscribes a wound or skin condition.

5. The system of claim 4 wherein the covering portion overlies essentially all of the base portion.

6. The system of claim 1 wherein the covering portion defines a pull tab adapted to be gripped by a user to assist in separating the covering portion from the base portion.

7. The system of claim 1 further comprising a removable protective cover over the first adhesive on the lower side of the base portion.

8. A reclosable wound or skin condition dressing and treatment system, comprising:
a covering portion having a lower side that defines an annular adhesive and an upper side comprising a clear or translucent semi-permeable layer, the covering portion spanning the opening of the annulus such that it fully covers the open area of the annulus, to cover a wound or skin condition located in the open area while keeping the wound or skin condition visible through the covering portion.

9. The system of claim 8 wherein the adhesive comprises a hydrocolloid adhesive.

10. The system of claim 8 wherein the covering portion circumscribes a wound or skin condition.

11. The system of claim 8 wherein the covering portion defines a pull tab adapted to be gripped by a user to assist in separating the covering portion from the skin.

12. The system of claim 8 further comprising a removable protective cover over the adhesive.
